(19) United States
(12) Patent Application Publication
(10) Pub. No.: US 2019/0246636 A1
(43) Pub. Date: Aug. 15, 2019

(54) Redox Active Metal/Metal Oxide Composites For Antimicrobial Applications

(71) Applicant: AGENCY FOR SCIENCE, TECHNOLOGY AND RESEARCH, Singapore (SG)

(72) Inventors: Yugen ZHANG, Singapore (SG); Guangshun Yl, Singapore (SG)

(21) Appl. No.: 16/335,243

(22) PCT Filed: Sep. 15, 2017
(86) PCT No.: PCT/SG2017/050463

§ 371 Date: Mar. 20, 2019

(30) Foreign Application Priority Data
Sep. 20, 2016 (SG) 10201607856T

(51) Int. Cl. A01N 25/26; A01N 59/16; C09D 5/14; C09D 1/00; A01N 25/08; A61L 2/232; C23C 22/60

(52) U.S. Cl. CPC A01N 25/26; A01N 59/16; C09D 5/14; C23C 22/60; A01N 25/08; A61L 2/232; C09D 1/00

(57) ABSTRACT

The invention relates to a method of preparing a metal oxide/metal composite, comprising depositing a metal oxide from a dispersion in a liquid on a metal surface; or depositing a metal oxide in the presence of a metal from a dispersion in a liquid on a substrate; or depositing a metal oxide from a metal salt solution on a metal substrate. The metal oxide/metal composites obtained by the process show synergistic antimicrobial activity due to release of high concentrations of redox active species (ROS) at the metal oxide/metal heterojunction. The invention also relates to use of the metal oxide/metal composite as an antimicrobial coating.

### TECHNICAL FIELD

The present invention generally relates to methods of preparing antimicrobial, redox active metal oxide/metal composite materials which have extraordinary microbial killing activity, due to the high redox activity which generates higher reactive oxygen species (ROS) concentrations than the individual components. It also relates to the materials as such and the use thereof in antimicrobial applications.

### BACKGROUND ART

Microbial infection is one of the most serious concerns for many commercial applications, such as medical devices, hospital surfaces, textiles, packaging, electrical appliances, filters and public surfaces. To create clean antimicrobial surfaces with long-term stability and activity has tremendous applications including almost all aspects of our daily life, from medical devices to construction surfaces. Currently, organic molecular antimicrobial agents, such as triclosan and biguanides, are standard ingredients in consumer care products as antiseptics, disinfectants and preservatives to inhibit microbial growth for preventing infections.

However, these conventional antimicrobial agents may cause serious concerns due to their toxicity to environment and potential resistance in microbes. Alcohol based hand sanitizers and surgical scrubs commonly used in hospital settings may cause skin irritation and dehydration. Bleach detergents are strong oxidants which kill microbial very efficient, however, they also cause serious environmental effects due to the irritating smell and harmful residuals.

Some metal or metal oxides, such as silver, zinc oxide and titanium oxide particles have been used as antimicrobial ingredients in various products or in antimicrobial surface coatings. However, these materials also have limitations such as heavy metal contamination/toxicity (Ag) or low microbial killing efficacy (ZnO/TiO₂) and uncertain nano toxicity.

From a mechanistic point of view, organic antimicrobials mostly interact with specific targets to dysfunction microbial organisms. They often cause resistance and/or toxic chemicals are released during the action. The mechanism of the activity of inorganic particles is not fully clarified, while commonly accepted mechanisms include: (1) direct contact of nanoparticles (NPs) with cell walls, resulting in destructing of bacterial cell integrity which is size dependent; (2) liberation of antimicrobial ions which are based on dissolved metals or released metal ions or (3) formation of reactive oxygen species (ROS). ROS typically generate from defects of metal oxide lattice or UV illumination. The ROS concentration is can be relatively low and less efficient.

Therefore there is a need in the art to increase the ROS releasing level of inorganic materials that have a use in antimicrobial applications, because this increase may be a very effective way to enhance their antimicrobial efficacy without causing other negative impacts. Hence there is a need for making inorganic materials that show such increase in ROS release. The inorganic materials should be environmentally friendly in their use and disposal and show long term stability.

There is also a need for making materials showing such increase in ROS release in a simply and scalable way.

### SUMMARY OF INVENTION

According a first aspect of the invention a method of preparing an antimicrobial oxide/metal composite material comprising the steps of (a) preparing a depositing medium comprising a metal oxide or a metal salt in a liquid; and b1) depositing the metal oxide from a dispersion in the liquid on a metal surface; or b2) depositing the metal oxide in the presence of a metal from a dispersion in the liquid on a substrate; or b3) depositing a metal oxide from a metal salt solution on a metal substrate; and c) separating the depositing medium from the formed composite material has been found.

Advantageously, the metal oxide/metal composite material obtained in the methods according to the invention shows high redox activity and a high release rate of ROS from the metal oxide/metal composite. The high reactive oxygen species (ROS) concentrations are higher than those of the individual components and lead to significantly higher and synergistically increased microbial killing activity. The heterojunction between the metal oxide/metal dual components generates a very high redox activity. Further advantageously, the method steps are simple and can be easily scaled up in commercial fabrication.

According to certain embodiments the metal oxide and metal can be chosen be of an environmentally friendly (“green”) type which advantageously leads to the avoidance of contamination by heavy metals or release of toxic substances while still achieving the improved antimicrobial activity.

According to a second aspect of the invention there is provided the antimicrobial composite material comprising at least one metal component and one metal oxide component with heterojunction obtained according to the method of the invention.

The materials are novel redox active metal/metal oxide composites which have very high ROS releasing level and improved microbial killing property. Advantageously the new materials can be used as additives in many consumer care, healthcare and cosmetic products. They can also be applied as surface coatings to create long term self-disinfecting surfaces including both hard surfaces and fabrics or textiles. The inorganic antimicrobial materials are clean and safe, stable and scalable in processing, and have broad range of applications. Advantageously, these composite materials have excellent long-term stability and antimicrobial activity without releasing any harmful chemicals. A novel antimicrobial composite material in layered structure or particle form comprising a metal oxide/metal composite wherein the metal oxide is selected from zinc oxide, iron (III) oxide or iron (II) oxide and the metal is selected from zinc or iron; and comprising at least one metal component and one metal oxide component with heterojunction has been further provided which shows the above mentioned advantages.

According to a third aspect of the invention, there is also provided the use of antimicrobial materials according to the invention for the killing of bacteria.

According to a fourth aspect of the invention, there is provided a method comprising the step of exposing a surface coated with the antimicrobial material according to the invention to the bacteria.

Definitions

The following words and terms used herein shall have the meaning indicated:

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “composite material (also called a “composition material” or in short form “composite” which is also the common name) refers to a material made from two or more constituent materials with significantly different physical or chemical properties that, when combined, produce a material with characteristics different from the individual components.

As used herein, the term “heterojunction” refers to the interface that occurs between two components of dissimilar type, such as metal and metal oxide.

As used herein, the term “antimicrobial” or “antimicrobial activity” refers to the capability to kill microorganisms or control the growth of microorganisms.

As used herein, the term “high temperature growth reaction” or “high temperature growth method” refers to a synthesis reaction which includes crystallizing substances from high-temperature solutions.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Unless specified otherwise, the terms “comprising” and “comprise”, and grammatical variants thereof, are intended to represent “open” or “inclusive” language such that they include recited elements but also permit inclusion of additional, not recited elements.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Non-limiting embodiments of the invention will be further described in greater detail by reference to specific examples, which should not be construed as in any way limiting the scope of the invention.

According to a first aspect, there is provided a method of preparing an antimicrobial metal oxide/metal composite material comprising the steps of: a) preparing a depositing medium comprising a metal oxide or a metal salt in a liquid; and b1) depositing the metal oxide from a dispersion in the liquid on a metal surface; or b2) depositing the metal oxide in the presence of a metal from a dispersion in the liquid on a substrate; or b3) depositing a metal oxide from a metal salt solution on a metal substrate; and c) separating the depositing medium from the formed composite material.

The composite material comprises a metal oxide and a metal. The metal of the oxide and the metal of the metal component may be chosen independently. Accordingly the metal of the metal and the metal oxide can be the same or different. The metal oxide may be selected from zinc oxide, iron (III) oxide, iron (II) oxide, cobalt (III) oxide, cobalt (II) oxide, nickel (III) oxide, nickel (II) oxide, copper (II) oxide or copper (I) oxide, manganese (II) oxide, titanium oxide, chromium (III) oxide, chromium (II) oxide, vanadium (V) oxide, aluminum (III) oxide, germanium dioxide or tin dioxide or a mixture of these oxides. Zinc oxide or iron (III) oxide may be particularly mentioned. Zinc oxide may be most preferred. The metal may be selected from a group consisting of zinc, aluminum, iron, cobalt, nickel, copper, manganese, chromium, vanadium, germanium, and tin. A mixture of these metals or alloys of this metals are also included. Zinc and iron may be particularly mentioned.

Metal/metal oxide composites of the invention may for instance be composed of combinations of metals and metal oxides, including Fe, Fe₂O₃, FeO, Fe₃O₄, Co, CoO, Co₂O₃, Ni, NiO, Cu, CuO, Zn, ZnO, Mn, Mn₂O₃, Ti, TiO2, Cr, Cr₃O₄, V, V₂O₅, Al, Al₂O₃, Ge, GeO₂, Sn, SnO₂. The composite materials have at least two components which contain at least one metal oxide and one or more metals. Specifically preferred combinations of metal oxide/metal composites comprise the following combinations: zinc oxide/iron, zinc oxide/aluminum and iron (III) oxide/zinc. The composites may present a heterostructure where two or more components have heterojunctions. The heterojunction between the metal and metal oxide components will have very high redox activity, which could generate several orders higher ROS concentration than individual components. Therefore, the hetero-structured redox active composites have extraordinary microbial killing or controlling activity.

The composite material shows antimicrobial activity. The antimicrobial metal oxide/metal composite material according to the invention may show an antimicrobial activity from the release of reactive oxygen species (ROS). The composites may show a higher ROS release than the individual components such as the metals and metal oxides individually. The material may therefore show a synergistic effect in the release of ROS.

In this regard they may show strong antibacterial activities against both Gram-positive and Gram-negative bacteria. As bacteria that can be inhibited or killed the following may be particularly mentioned: Escherichia coli, Salmonella, Listeria monocytogenes, and Staphylococcus aureus.

The method according to the invention to prepare the antimicrobial composite materials comprises three steps: a), b) and c). The steps may usually be performed in the order of a), b) and c). The process may comprise others steps.

In step a) a depositing medium comprising a metal oxide or a metal salt in a liquid is prepared. The liquid may be water, an alcohol or mixtures thereof. The alcohol may be a primary aliphatic alcohol or secondary aliphatic alcohol. Aliphatic alcohols, such as ethanol, may be particularly mentioned.

Step a) may comprise the dissolution or dispersion of a metal oxide in a liquid. The dispersion may be supported by the use of ultrasonic. The choice of the liquid is not crucial. Preferred liquids may be water, or a polar, organic solvent, such as for instance ethanol, methanol, acetone, methyl ethyl ketone, isopropanol, n-propanol, acetonitrile, DMSO (dimethyl sulfoxide) or DMF (dimethyl formamide); or mixtures thereof. The metal oxide may be used as powder or micro or nano-particulate material. The particle size is not crucial, but typical particle sizes that can be mentioned are about 25 nm to 10 μm, 200 nm to 10 μm, 400 nm to 5 μm, 500 nm to 1 μm, or 300 nm to 700 nm.

It may be chosen from any metal oxide mentioned as part of the metal oxide/metal composite. Zinc oxide or iron(III)oxide can be particularly mentioned.

It may be used in a concentration of 0.01 to 1 g per mL of liquid. A concentration of 0.05 to 0.3 g/mL may be preferred. Concentrations of 0.03, 0.07, 0.5 or 0.7 g/mL are also suitable.

Alternatively step a) may comprise the dissolution of a metal salt in the liquid wherein the liquid becomes a solvent for the metal salt. Preferred liquid solvents may be water, or a polar, organic solvent, such as for instance methanol or ethanol; or mixtures thereof. The metal salt shall be soluble in the liquid. Typical metal salts that can be mentioned include chlorides, nitrates or sulphates. Zinc chloride, zinc nitrate and zinc sulphate may be particularly mentioned. The dissolved metal salt will be used in the later steps to form the metal oxide by precipitation, oxidation or reduction. The metal salt may be used in concentrations of 0.1 to 4 molar, preferably 0.3 to 0.7 molar.

Step b) has three alternatives: step b1), b2) or b3). At least one of the steps is performed according to the method of the invention.

In step b1) the dispersion of the metal oxide prepared in step a) is used to deposit the metal oxide on a metal surface. The metal surface can be any surface of a metal such as the surface of a metal particle, metal powder or metal sheet or any other metal article. The metal oxide is directly deposited on the metal surface to create the inventive heterojunction between the deposited oxide and the metal surface. In one embodiment the dispersion of the metal oxide in the liquid is casted on the surface of a metal and in step c) the liquid is removed to deposit the metal oxide. Removal can be by evaporation.

In step b2) the dispersion of the metal oxide prepared in step a) is used to deposit the metal oxide in the presence of a metal on a substrate. The substrate does not need to be a metal. The metal which is present is preferably in easy depositable form such as in powder or particle form. The metal is selected from the metals mentioned above for the metal oxide/metal composites. During the deposition the metal and the metal oxide are deposited together in way that they form the heterojunction after step c) by being in direct contact. In one embodiment the dispersion of the metal oxide is deposited together with a dispersed metal powder. A metal oxide powder and a metal powder may be premixed before dispersion in the liquid. The metal powder preferably may have a particle size of about 0.1 to 100 μm, more preferably between about 0.7 to 20 μm, and in step c) the solvent is removed to deposit the metal oxide and the metal. Such metal powders are commercially available. The metal particle and powders that can be used in step b2) may have a particle size of about 0.1 to 100 μm. Other sizes may also be suitable, e.g. about 0.5 to 500 μm, about 1 to 10 μm, 0.7 to 20 μm, or about 5 to 50 μm. Powders with spherical shape or smooth surface may be preferred. However, micro or nanoparticles of 1 to 100 nm, preferably 10 to 50 nm, may also be used.

In step b3) the dissolution of the metal salt prepared in step a) is used to deposit a metal oxide on a metal substrate. The metal substrate can be any metal material such as particles of a powder, a metal article etc. The metal substrate may be a metal particle, a metal micro particle or metal nanoparticle. Particles with spherical shape or smooth surface may be preferred. The particle may have a size of about 0.01 to 100 μm, preferably between about 0.05 to 50 μm, more preferably between about 0.1 to 10 μm.

In one embodiment of step b3) the metal oxide is deposited by high temperature growth reaction from a metal salt solution on the metal substrate and wherein the high temperature growth synthesis step is carried out between about 50° C. and 300° C. Preferably the high temperature growth is performed in an aqueous solution. The high temperature growth synthesis step may or may not be a hydrothermal reaction. Preferably the thermal hydration step is carried out between about 70° C. and 120° C., more preferably between about 80° C. and 100° C. A temperature of about 90 to 97° C. can be particularly mentioned. In this embodiment the metal salt solution can be made from typical metal salts including nitrates, chlorides or sulphates. Zinc nitrate may be particularly mentioned. Preferably a base is added. Preferred bases include nitrogen bases, such as ammonia (NH₃) or hexamethylenetetramine (HMT). Water, particularly deionized water may be used as the solvent optionally in pre-heated form. The high temperature growth reaction is usually carried out between 2 minutes to 10 hours. The reaction time can be about 5 minutes to 40 minutes, or 5 minutes to 30 minutes, preferably 10 to 20 minutes. The metal oxide, especially zinc oxide can be grown in the form of layers. The concentration of the metal salt in the chemical solution can be varied widely; preferably it is about 10 mM to 1 M, or about 100 to 500 mM or about 200 to 400 mM. The deposition may take place on a metal substrate. The particles may have a size of about 0.01 to 100 μm, preferably between about 0.05 to 50 μm, more preferably between about 0.1 to 10 μm. The base, such as ammonia, may be used at a concentration of about 0.01 M to 1 M, or about 100 to 500 mM. The pH is usually chosen between pH 7 and 12, or between 9 and 10.5.

In another embodiment of step b3) the metal oxide is deposited by precipitation of the metal oxide from a metal salt solution on a metal substrate, such as a metal particle, by reaction with a base. The reaction may be run at about room temperature, at about 15° C. to 30° C. The particle may have a size of about 0.01 to 100 μm or about 50 nm to 10 μm, preferably between about 0.05 to 60 μm, more preferably between about 0.1 to 15 μm. A strong inorganic base may be used to precipitate the metal oxide from the metal salt solution on the metal substrate surface. NaOH and KOH may be particularly mentioned as suitable bases. The metal oxide prepared may be zinc oxide. In this embodiment the metal salt solution can be made from typical metal salts including nitrates, chlorides or sulfates. The use of a Zn(NO₃)₂ solution may be particularly mentioned. The precipitation reaction may be carried out between about 1 to 5 hours. The reaction time can be about 1 to 4 hours, more preferably in the range of about 1 to 3 hours. In this case the metal oxide, especially zinc oxide, can be grown in the form of fine pillars of nano-size needles on the metal substrate. The concentration of the metal salt can be varied widely; preferably it is about 50 mM to 2 M, or about 100 to 500 mM or about 200 to 400 mM. The concentration of base can be varied widely; preferably it is about 500 mM to 5 M, or about 1 to 4 M or about 1.5 to 3 M. The needles obtained have a length of about 500 to 2000 nm, preferably 700 nm to 1200 nm.

In step c) of the method according to the invention the depositing medium is separated from the formed composite material. Typical separation techniques can be used for the separation which includes filtration, evaporation, rinsing, washing or drying steps.

In one embodiment after step b1) or b2) the metal oxide dispersion medium's liquid is evaporated to form the metal oxide on the metal surface. This evaporation step can be repeated after repeating step b1). It is may be repeated 2 to 3 times. Repetition of step b1) or b2) combined with c) may lead to a uniform coating of a coating layer with heterojunction between oxide layer and metal surface.

In another embodiment after step b3) the metal substrate, such as the powder or particle, will be separated from the liquid by filtration or centrifugation. Thereafter the oxide modified metal substrate may be washed with water or organic solvents, such as for instance alcohols. The washing steps can be repeated 2 to 4 times to ensure the removal of impurities from the deposition medium. The final metal substrate may be dried at room temperature or using heat. A long-term stable composite can be achieved after step c). In one embodiment the final metal substrate is a metal particle or powder modified with the metal oxide.

All steps of the method according to the invention may be performed at ambient temperature (about 20° C. to 27° C.), if not mentioned differently above.

According to a second aspect of the invention an antimicrobial composite material comprising a metal oxide/metal composite and wherein at least one metal component and one metal oxide component with heterojunction are obtained according to the method of the invention as described above. The material may comprise a metal oxide/metal composite that is an iron (III) oxide/zinc, zinc oxide/zinc, zinc oxide/aluminum or zinc oxide/iron composite. The material releases high ROS at an effective level of antimicrobial activity that can be determined by the method described herein. The material may be able to release ROS concentrations of at least about 1 μmol/cm² of its surface within 5 minutes. In 5 hours it may release ROS concentrations of about 1 μmol/cm² to 1000 μmol/cm², preferably about 3 μmol/cm² to 100 μmol/cm², more preferably 5 μmol/cm² to 50 μmol/cm² and most preferably 10 μmol/cm² to 35 μmol/cm².

The metal/metal oxide composite of the material according to the invention may comprise 1 to 85%, more preferably 3 to 35%, more preferably 5 to 40%, by weight of the metal oxide. The rest may be the metal or the metal or an admixture with other components comprising the metal. The ratio of metal to metal oxide is about 4:1 to 1:5. In the case of particles the ratio may depend on the particle size and is about 4:1 to 2:1 for micrometer size particles of 1 to 100 μm and about 1:2 to 1:5 for nanometer size particles of 10 to 100 nm.

The material may comprise other components than the metal and metal oxide, such as fillers, colorants, carriers, mixtures and alloys of other metals etc. which are known in the field of use. The amount of the other materials in the material is not critical and can be for instance between 0.5 to 99.5% by weight, preferably 5 to 40% by weight. The composite material may further be obtained in various forms including, but not limited to, an alloy or a doped form of the metal, a core-shell or layered structure, a coating or in co-crystallization form or a mixture of the deposited components.

However, it is important that the at least one metal and the metal oxide form a heterojunction in the composite material to achieve the antimicrobial activity. The heterojunction in the material may be in direct contact with the medium that needs to be treated for antimicrobial activity.

The material may be in form of a metal particle or metal article of any geometrical form with metal oxide deposited thereon, preferably in the metal article is in a metal sheet form. The metal oxides deposited on the particle or metal article may be found in various forms which may support the antimicrobial activity. They can be deposited as flat layer, in form of nano needles or rods as well as flower like, high surface geometries.

If particles are obtained, they can have a size of about 0.01 to 100 μm, preferably between about 0.05 to 50 μm, more preferably between about 0.1 to 10 μm. They can be in core/shell structure wherein the metal is comprised in the core of the particle. The particle core may be round or spherical, although the invention is not restricted to such geometries. A round or spherical form may be more suitable to have a basis for the deposition of the metal oxide in layers or pillars of nano structures. The metal oxide may be comprised in the shell of the particle. The shell may form a layer around the core. Layered particles may be obtained wherein step b2) comprises the precipitation of a metal oxide on the particle core. Alternatively the shell may comprise a pillar of nano- or microstructured metal oxides such as for instance needles or rods. Nano needles of a size of about 1 nm to 3 μm, preferably about 50 nm to 1000 nm, most preferably 5 nm to 100 nm can be mentioned. Pillar particles with nano needles may be obtained wherein step b2) comprises the deposition of a metal oxide on the particle core.

The metal articles can be based on a metal selected from the group consisting of zinc, aluminum, iron, cobalt, nickel, copper, manganese, chromium, vanadium, germanium, or tin and their alloys. As sheets there can be particularly mentioned iron and steel sheets, aluminum sheets, galvanized and aluminized steel sheets, stainless steel sheets, or zinc metal sheets. The metal of the metal article can also be selected from the group consisting of steel, zinc, zinc based alloys, zinc coated steel, zinc aluminum alloy coated steel, aluminum and aluminum alloy. However, the geometry of the metallic article is not crucial and can be adapted to the use field of the antimicrobial material.

According to another embodiment the antimicrobial composite material is a mixture of metal particles and metal oxide particles deposited according to the method of the invention. The particles can have a size of about 0.01 to 100 μm, preferably between about 0.05 to 50 μm, more preferably between about 0.1 to 10 μm. They can be mixed in various ratios from 5:95 to 99:5 weight percent; preferably they are mixed at 30:70 to 70:30 weight percent and most preferably 40:60 to 60:40 weight percent. The metal may however be used in excess such as about 60, 70, 80 or 90% by weight.

According to one embodiment of the invention an antimicrobial composite material may be in layered structure or particle form comprising a metal oxide/metal composite and comprising at least one metal component and one metal oxide component with heterojunction obtained according to the method of the invention.

According to another embodiment the composite material is directly obtained as such from the method according to the invention. In this embodiment the composite is directly made as part of the composite material by steps a) to c) and not introduced by other means such as for instance spraying of composite particles or similar coating methods using particles.

According to another aspect of the invention the material is obtainable according to the method of the invention, but may be made by a different process resulting in substantially the same material with the above mentioned technical features of the materials made according to the process of the invention. Specifically an antimicrobial composite material in layered structure or particle form comprising a metal oxide/metal composite wherein the metal oxide is selected from zinc oxide, iron (III) oxide or iron (II) oxide and the metal is selected from zinc or iron; and comprising at least one metal component and one metal oxide component with heterojunction is novel per se. It is part of the invention as another aspect of the invention. This material has the same features as described above for the materials in layered structure or particle form comprising a metal oxide/metal composite and comprising at least one metal component and one metal oxide component with heterojunction obtained according to the method of the invention.

According to a third aspect of the invention, there is also provided the use of antimicrobial materials according to the invention for the killing or controlling of microorganisms. The use may be limited to a use not related to the medical treatment of humans or animals such as the coating of inanimate object or personal hygiene applications. A use for coating an inanimate object or for cleansing of the external surface of a human or animal body may be particularly mentioned. “A composition cleansing of the external surface of a human or animal body” refers to a composition in the form of a leave-on or wash-off format meant for cleaning or disinfecting topical areas e.g. skin and/or hair of mammals, especially humans. Such a composition includes any product applied to a human body for also improving appearance, cleansing, odour control or general aesthetics. The materials may be further used for the preparation of a medicament for the treatment of bacterial infections. Such medicament can for instance be a topical ointment.

Microorganisms can be controlled by the materials in a variety of media by contacting an effective amount of the antimicrobial material with a microorganism in the medium. A convenient medium is an aqueous medium. Contacting the skin or other parts of a mammal or a surface of an article that is to be disinfected with an effective amount of the antimicrobial material would also be expected to control microorganisms. The antimicrobial material of the present invention controls a broad spectrum of microorganisms. The material has been found to be especially useful in controlling bacteria. By the term “bacteria” is meant eubacteria and archaebacteria. Eubacteria include fermicutes, gracilicutes and ternicutes. Gracilicutes include gram-negative, facultatively anaerobic rods. Gram-negative, facultatively anaerobic rods include Enterobacteriaceae. Enterobacteriaceae include Klebsiella and Escherichia. Klebsiella include Klebsiella pneumoniae and Escherichia include Escherichia coli. Fermicutes include the group gram-positive cocci, and the group endospore-forming rods and cocci. Gram-positive cocci include Micrococcaceae. Micrococcaceae include Staphylococcus and Staphylococcus includes Staphylococcus aureus. Endospore-forming rods and cocci include Bacillaceae. Bacillaceae includes which includes Bacillus circulans. All references herein to bacteria are in accordance with Bergey's Manual of Systematic Bacteriology, Williams & Wilkens, 1st ed. Vol. 1-4, (1984).

According to fourth aspect of the invention, a use for killing or controlling microorganism, such as bacteria, by exposing a surface coated with the antimicrobial material according to the invention to the medium comprising the microorganism or bacteria is provided. In this regard metal sheets with metal oxides deposited thereon according to the invention may be particularly mentioned.

### EXAMPLES

Non-limiting examples of the invention and a comparative example will be further described in greater detail by reference to specific Examples, which should not be construed as in any way limiting the scope of the invention.

Examples of metal oxides coated metal surfaces including ZnO/Zn, ZnO/Al, Fe₂O₃/Zn and ZnO/steel surfaces, as well as ZnO/Zn core-shell particle, ZnO+Zn conjugates are given to demonstrate the inventive concept.

Materials

Commercially available Zn powder with a particle size of 1 to 10 or 50 μm was purchased from Sigma.

Methods

Surface Characterization: The surfaces of the samples were characterized by SEM (JEOL JSM-7400E), TEM (FEI Tecnai F30) and XRD (PANalytical X-ray diffractometer, X'pert PRO, with Cu Kα radiation at 1.5406 \acute{Å}). Prior to SEM, the samples were coated with thin Pt film using high resolution sputter coater (JEOL, JFC-1600 Auto Fine Coater).

Bacterial growth conditions and sample preparation: Tryptic soy broth (TSB) was purchased from BD Diagnostics (Singapore) and used to prepare the broths according to the manufacturer's instructions. Gram-negative bacteria E. coli (ATCC No. 8739) was purchased from ATCC (U.S.A) and re-cultured according to the suggested protocols. Prior to bacterial experiment, bacterial cultures were refreshed on nutrient agar from stock. Fresh bacterial suspensions were grown overnight at 37° C. in 5 ml of TSB. Bacterial cells were collected at the logarithmic stage of growth and the suspensions were adjusted to OD600=0.07.

JIS killing efficacy testing: The tested bacteria were suspended in 5 mL of respective nutrient broth and adjusted to OD600=0.07. The solution was further diluted 10² times for antibacterial testing. In order to cover the surface, 150 μL of cell suspensions was placed on the surfaces. After incubation at 37° C. with the surfaces, the respective cell suspensions were washed and diluted, and each dilution spread on two nutrient agar plates. Resulting colonies were then counted using standard plate counts techniques, and the number of colony forming units per mL was calculated. The number of colony forming units was assumed to be equivalent to the number of viable cells in suspension.

ROS testing method (see X. Hu, K. G. Neoh, J. Zhang, and E.-T. Kang, J. Colloid Interf. Sci., 2014, 417, 410): The ROS was determined by luminol-based chemoluminance assay. In brief, the substrates were placed in a 24-well microplate, and 1 ml of 0.2 M NaOH solution containing 5 mM luminol was added to each substrate in the dark. The chemoluminance was measured with a microplate reader (Tecan Infinite, Switzerland) at 1, 2, 4, 8 and 24 h. The ROS density was calculated based on a standard curve prepared by using the Fenton reaction (addition of predetermined amount of 50 mM hydrogen peroxide and 0.02 M ferrous sulfate into the 5 mM luminol solution).

### EXAMPLES

### Example 1

### ZnO-Metal Foil with Antibacterial Properties

ZnO coatings on Zn, Al, Fe substrates have been prepared. 0.1 g ZnO powder with a particle size of 200 to 500 nm was added in 1 mL ethanol, and dispersed by ultrasonic for 5 min. 100 μL of solution was dispersed onto the surface of Zn, Al and Fe substrates with a dimension of 2×2 cm. After evaporation of ethanol, another 100 μL of solution was applied. After evaporation of ethanol, a uniform ZnO coating was formed on the surface.

The sub-micrometer ZnO powder deposited onto different substrates (Zn, Al, and steel) formed a ZnO coating (FIG. 1). The antibacterial activities of the coated metals were evaluated using JIS Z 2801/ISO 22196 method, which is well recognized as industrial standard for the assessment of antibacterial surfaces.

As shown in FIG. 2, all bacterial cells of E. coli were killed (with logarithmic reduction greater than 8) on ZnO coated ZnO/Zn, ZnO/Al and ZnO/Fe surfaces after 24 h incubation. As control, all bacteria on flat metal foils (Zn, Al, Fe) kept on growing during incubation indicating non-biocidal property under the testing conditions. In addition, a ZnO coating on a glass surface also shows insufficient biocidal property by using the same evaluation method.

These results indicate that the ZnO/metal composites possess superior antibacterial properties as compared to single component of ZnO or metals. To investigate the antibacterial mechanism of ZnO/metal composites, the Zn²⁺ ion release level and the reactive oxygen species (ROS) level of several ZnO coating surfaces including Zn, Ti and glass were measured. Zn²⁺ ion release level was monitored by ICP-MS and the reactive oxygen species (ROS) level was measured by chemoluminescence method (FIG. 3).

From FIG. 3, the leaching of Zn²⁺ ions from ZnO coatings on various substrates is shown to be of a similar level. However, ROS concentration of a ZnO/Zn composite is much higher than the rest. This result suggests that the ROS release is the main reason for combating bacteria. Zn, ZnO/glass and ZnO/Ti have no bactericidal property (logarithmic reduction<1) as evaluated by using JIS method.

### Example 2

### ZnO—Zn Particles with Antibacterial Properties

ZnO/Zn core/shell particles were prepared using two different ways from commercially available zinc powder (Sigma). The as-received Zn powder has spherical shape with smooth surface (FIG. 4A)

For the growing of ZnO nanoneedles, Zn powder was treated in a solution of KOH and Zn(NO₃)₂. 5 ml of 0.5 M Zn(NO₃)₂ aqueous solution and 5 ml of 4 M KOH aqueous solution were filled in a reaction tube. 1 g of Zn particle was added. The mixture is kept at room temperature for 2 hours under gentle agitation. After that, the powder was washed with water 3 times and ethanol 3 times, dried in vacuum and stored for future use. When Zn powder was treated with KOH/Zn(NO₃)₂ solution for 2 hours, ZnO pillars were grown on the Zn particle surface (FIG. 4B).

ZnO—Zn particles were also prepared by high temperature growth reactions. To a 0.1 M aqueous ZnSO₄ solution 30% NH₄H₂O were added until the pH reached 10. Then 0.3 to 1 g Zn powder were added and the reaction mixture heated to 95° C. for 15 min. ZnO was growing on the Zn particles surface to form shell layer (FIG. 4C). XRD studies confirmed the formation of ZnO on Zn, as shown in FIG. 4(D).

To test the antibacterial properties of these both particle types, 0.02 g of the obtained particles were dispersed in ethanol, and coated onto a glass slides with dimension of 2.5 cm×2.5 cm. As comparison, blank glass slides, glass slides coated with 0.02 g Zn powder, 0.02 g ZnO powder and a mixture of 0.01 g Zn powder with 0.01 g ZnO powder were also tested.

The antibacterial properties of the surfaces were evaluated by the JIS method. As showed in Table 1 and FIG. 5, surfaces with Zn/ZnO composite all showed good antibacterial properties, while the rest of the surfaces did not show antibacterial properties under the same testing conditions. Zn/ZnO composite in particle form also show good antibacterial property which is similar to the Zn/ZnO flat surface. In contrast, single component coating with Zn or ZnO particle alone does not show bactericidal property.

[Table 1] shows the antibacterial properties of glass slides coated with different particles (for entries 5 to 7, all bacterial cells were killed with the log reduction great than 8).

TABLE 1

| | | log |
|---|---|---|
| Item | Coating composition on glass | reduction |
| 1 | Control (blank Petri dish) | 0 |
| 2 | Blank glass slides | 0 |
| 3 | Zn powder coated on glass slides | 0 |
| 4 | ZnO powder coated on glass slides | 0 |
| 5 | ZnO/Zn core/shell particle (method 1) coated on | >8 |
| | glass slides | |
| 6 | ZnO/Zn core/shell particle (method 2) coated on | >8 |
| | glass slides | |
| 7 | Zn powder + ZnO powder coated on glass slides | >8 |

### Example 3

### Fe₂O₃—Zn Foil with Antibacterial Property

0.1 g Fe₂O₃ powder was added in 1 mL ethanol, and dispersed by ultrasonic for 5 min. 100 μL of solution was dispersed onto the surface of a Zn substrate (2×2 cm). After evaporation of ethanol, another 100 μL of solution was applied. After evaporation of ethanol, a uniform Fe₂O₃ coating was formed on the surface.

In addition to ZnO/metal composites, Fe₂O₃/Zn composite also demonstrates high bactericidal activity. As shown in FIG. 6, micron meter sized Fe₂O₃ particles were prepared and coated onto Zn foil surface. Antibacterial activity was then evaluated by using JIS method. As further shown in FIG. 6, all bacterial cells of E. coli were killed on Fe₂O₃/Zn with a logarithmic reduction>8, while Fe₂O₃ particles alone did not exhibit any bactericidal property.

In summary, it is shown in the examples that the metal-metal oxide composites, including ZnO/Zn, ZnO/Al, ZnO/Fe and Fe₂O₃/Zn, exhibit excellent antibacterial properties. The antibacterial property is due to the release of high concentration of ROS which may relate to active metal/metal oxide redox reactions.

### DESCRIPTION OF DRAWINGS

The accompanying drawings illustrate a disclosed embodiment or reaction scheme and serve to explain the principles of the disclosed embodiments. It is to be understood, however, that the drawings are designed for purposes of illustration of examples only, and not as a limitation of the invention.

FIG. 1 shows (A) the surface of the sub-micrometer particles after deposition of the oxide; (B) an XRD pattern of the ZnO powder of the example; (C) the surface of ZnO powder coated onto a glass substrate.

FIG. 2 shows the results of the antibacterial activity (against E. coli) measurement of ZnO particle coated on different substrates.

FIG. 3 shows (A) Zn²⁺ ion concentration in the TSB solution, and (B) the ROS concentration in the testing solution with ZnO coated surfaces.

FIG. 4 shows (A) an image of untreated Zn powder, and (B) an image of Zn powder treated in KOH/Zn(NO₃)₂ solution at room temperature, (C) an image of Zn powder treated with [Zn(NH₃)₄]²⁺ solution at 95° C. for 15 min, and (D) the XRD pattern of Zn powders before and after treatment (* are XRD peaks of ZnO).

FIG. 5 shows the results of the antibacterial properties (against E. coli) of ZnO/Zn core-shell particles as measured.

FIG. 6 shows an SEM image of Fe₂O₃ particles (A) and the results of antibacterial properties (against E. coli) of Fe₂O₃/Zn composite as measured.

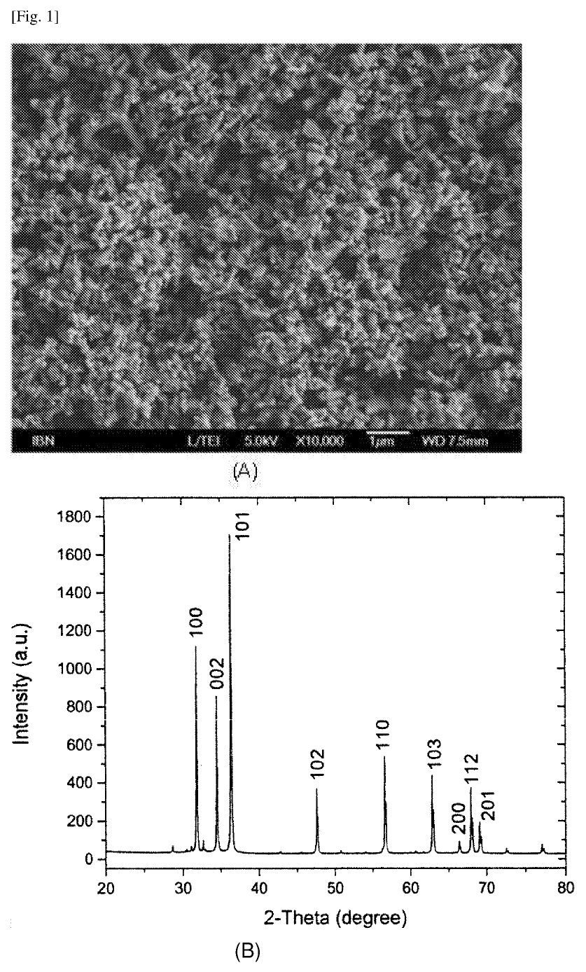

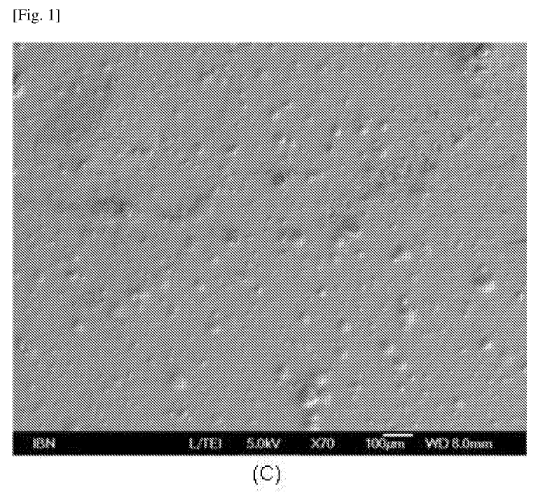

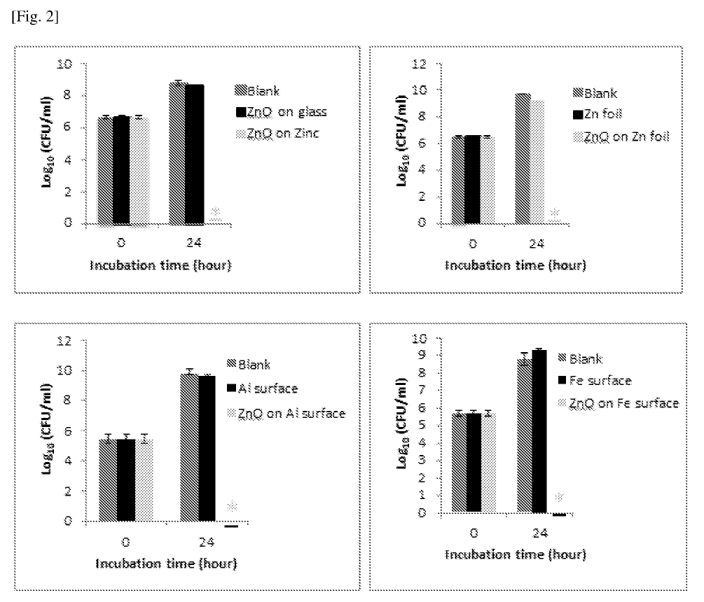

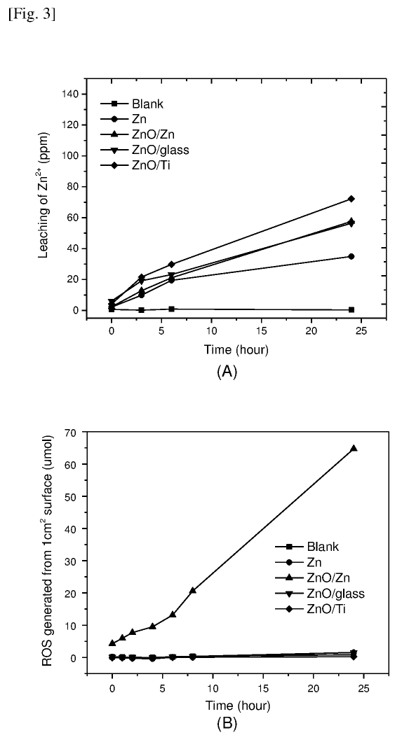

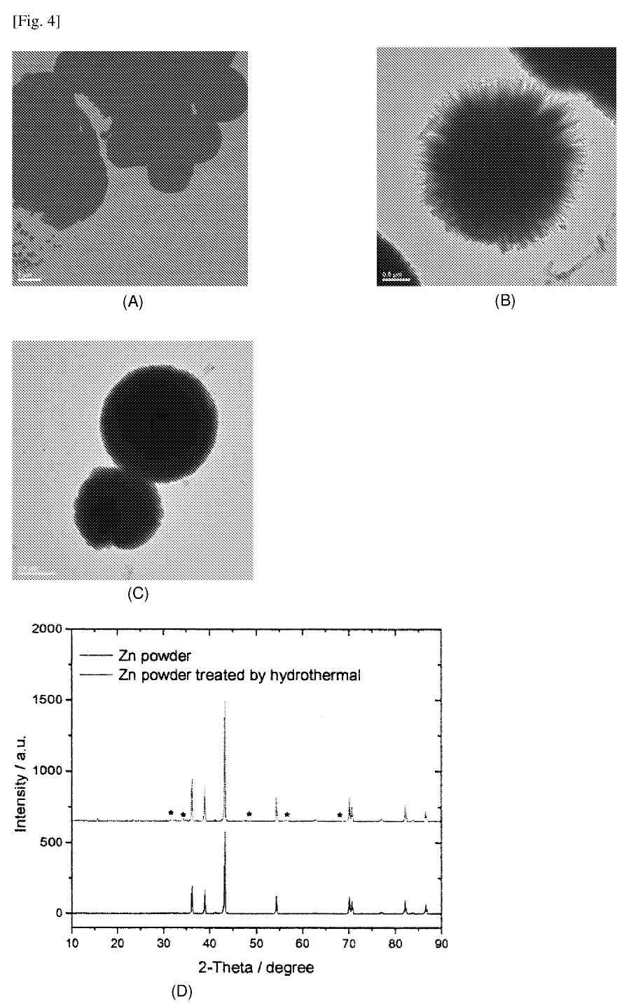

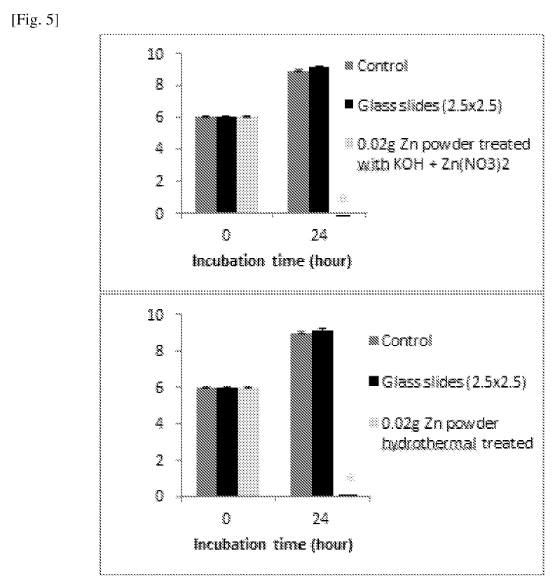

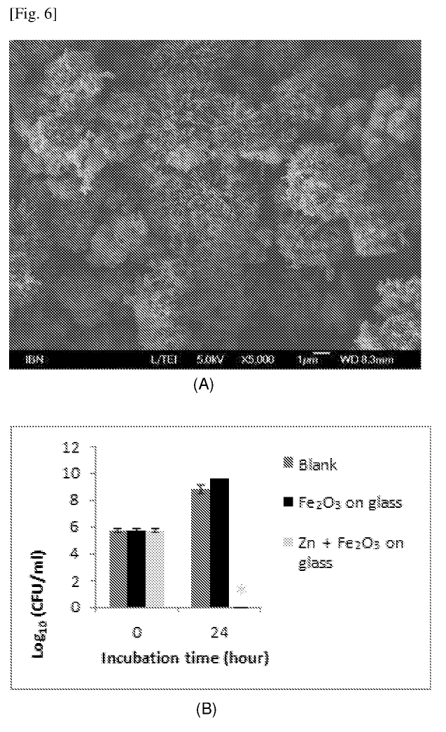

### INDUSTRIAL APPLICABILITY

The metal oxide/metal composite materials obtained by the method according to the first aspect of the invention exert anti-microbial activity. The activity is based in release of redox active species in high concentration. In this way they can be the basis for new environmentally friendly inorganic antimicrobial materials which are very stable and have long-term activity. The new materials are not size dependent and they are active in the size ranging from nanoscale to macroscale. The new materials could be used as additives in many consumer care, healthcare and cosmetic products. They can also be applied as surface coatings to create long term self-disinfecting surfaces including both hard surfaces and fabrics or textiles. The inorganic antimicrobial materials are clean and safe, stable and scalable in production and have a broad range of applications. They are susceptible to mass production by the inventive method. A scale up for water disinfection applications while avoiding the need for chemical treatment may be developed. The materials can also be recycled after use.

The new materials may replace common antimicrobial materials in the applications mentioned above.

It will be apparent that various other modifications and adaptations of the invention are available to the person skilled in the art after reading the foregoing disclosure without departing from the spirit and scope of the invention and it is intended that all such modifications and adaptations come within the scope of the appended claims.

1.-33. (canceled)

34. A method of preparing an antimicrobial metal oxide/metal composite material comprising:
a) preparing a depositing medium comprising a metal oxide or a metal salt in a liquid; and
b1) depositing the metal oxide from a dispersion in the liquid on a metal surface; or
b2) depositing the metal oxide in the presence of a metal from a dispersion in the liquid on a substrate; or
b3) depositing a metal oxide from a metal salt solution on a metal substrate; and
c) separating the depositing medium from the formed composite material.

35. The method according to claim 34, wherein the, metal oxide is selected from zinc oxide, iron (III) oxide, iron (II) oxide, cobalt (Ill) oxide, cobalt (II) oxide, nickel (III) oxide, nickel (II) oxide, copper (II) oxide or copper (I) oxide, manganese (II) oxide, titanium oxide, chromium (III) oxide, chromium (II) oxide, vanadium (V) oxide, aluminum (III) oxide, germanium dioxide, or tin dioxide, or mixtures thereof or
wherein the metal is selected from zinc, aluminum, iron, cobalt, nickel, copper, manganese, chromium, vanadium, germanium, or tin; or mixtures and/or alloys thereof.

36. The method according to claim 34, wherein in operation b1) the dispersion of the metal oxide in the liquid is casted on the surface of a metal and in operation c) the solvent is removed to deposit the metal oxide.

37. The method according to claim 34, wherein in operation b2) the dispersion of the metal oxide is deposited together with a metal powder, which preferably has a particle size of 0.1 to 100 μm and in operation c) the liquid is removed to deposit the metal oxide and the metal.

38. The method according to claim 34, wherein operations b1) or b2) and c) are repeated at least once.

39. The method according to claim 34, wherein the metal oxide is dispersed in the solvent by ultrasonic in operation a).

40. The method according to claim 34, wherein the liquid comprises an alcohol, or
wherein the alcohol comprises an aliphatic alcohol selected from primary aliphatic alcohol or secondary aliphatic alcohol, or
wherein the primary aliphatic alcohol comprises ethanol.

41. The method according to claim 34, wherein in operation b3) the metal oxide is deposited by high temperature growth reaction from a metal salt solution on a metal substrate and wherein the high temperature growth synthesis operation is carried out at a temperature of between about 50° C. and 300° C.

42. The method according to claim 41, wherein the metal substrate is a particle of a size of about 0.01 to 100 μm.

43. The method according to claim 41, wherein the metal oxide is zinc oxide which is deposited from a zinc salt solution.

44. The method according to claim 34, wherein in operation b3) the metal oxide is deposited by high temperature growth reaction from a metal salt solution on the metal particle in a layer form or
wherein the layer form deposition is carried out over a time period in the range of 5 minutes to 1 hour.

45. The method according to claim 34, wherein in operation b3) the metal oxide is deposited by precipitation of the metal oxide from a metal salt solution on the metal particle by reaction with a base or
wherein the base is NaOH or KOH or
wherein the metal oxide is zinc oxide or
wherein the metal salt solution is a Zn(NO₃)₂ solution.

46. The method according to claim 34, wherein in operation b3) the metal oxide is deposited by precipitation of the metal oxide from a metal salt solution on the metal particle in a pillar form.

47. The method according to claim 46, wherein the pillar form precipitation is carried out over a time period in the range of 1 to 5 hours, preferably in the range of 1 to 4 hours, more preferably in the range of 1 to 3 hours.

48. An antimicrobial composite material comprising a metal oxide/metal composite and wherein at least one metal component and one metal oxide component with heterojunction are obtained according to a method of preparing an antimicrobial metal oxide/metal composite material comprising:
a) preparing a depositing medium comprising a metal oxide or a metal salt in a liquid; and
b1) depositing the metal oxide from a dispersion in the liquid on a metal surface; or
b2) depositing the metal oxide in the presence of a metal from a dispersion in the liquid on a substrate; or
b3) depositing a metal oxide from a metal salt solution on a metal substrate; and
c) separating the depositing medium from the formed composite material.

49. A material according to claim 48, wherein the metal oxide/metal composite is an iron (III) oxide/zinc, zinc oxide/zinc, zinc oxide/aluminum or zinc oxide/iron composite.

50. An antimicrobial composite material in layered structure or particle form comprising a metal oxide/metal composite wherein the metal oxide is selected from zinc oxide, iron (III) oxide or iron (II) oxide and the metal is selected from zinc or iron; and comprising at least one metal component and one metal oxide component with heterojunction.

51. The material according to claim 48, wherein the material can release ROS concentrations of at least about 1 μmol/cm² of its surface within 5 minutes.

52. The material according to claim 48, wherein the material is in alloy, doping, core-shell or layered structure, coating or co-crystallization form or a mixture of the components.

53. The material according to claim 48, in particle form comprising a metal core and a metal oxide shell structure and wherein the core particle size is about 0.01 to 100 μm, preferably between about 0.05 to 50 μm, more preferably between about 0.1 to 10 μm or
wherein the metal oxide particle shell is in layer form, nano needle or pillar form.
